(19) United States
(12) Patent Application Publication
(10) Pub. No.: US 2026/0178177 A1
(43) Pub. Date: Jun. 25, 2026

(54) INTERACTIVE USER INTERFACE

(71) Applicant: Milan Madhavji, Mississauga (CA)

(72) Inventor: Milan Madhavji, Mississauga (CA)

(21) Appl. No.: 19/063,732

(22) Filed: Feb. 26, 2025

(30) Foreign Application Priority Data
Dec. 20, 2024 (CA) 3260271

(51) Int. Cl. G06F 3/04845; G16H 50/50

(52) U.S. Cl. CPC G06F 3/04845; G16H 50/50

(57) ABSTRACT

A computer system includes a display, at least one data storage device configured to access a volumetric dentoalveolar model representing a dentoalveolar state of a patient, at least one non-transitory computer-readable medium having instructions stored thereon which, when executed by at least one computer processor, cause the processor to access the model, provide a GUI in data exchange connection with the model, the GUI configured to include a plurality of data structures aligned in form and position with corresponding dentoalveolar structures of the model, display the model and the GUI on the display, in response to user input, select one data structure, and, update the GUI to provide at least one user control configured to adjust at least one of position, orientation, pan and scale of the model on the display relative to the one data structure in response to user input to the at least one user control.

The present invention relates to the presentation of diagnostic and treatment planning information to patients. More specifically, the present invention relates to the presentation of radiographic diagnostic information and dental treatment planning options to dental patients.

### BACKGROUND

In the field of dental care, accurate communication of diagnostic information between radiologists and patients is important for effective treatment planning and patient compliance. Traditionally, dental radiology involves the use of imaging techniques such as X-rays, cone-beam computed tomography (CBCT), and panoramic radiography to detect dental pathologies, assess bone structure, and evaluate oral health. However, while the acquisition and interpretation of radiographic data by professionals have advanced significantly, the presentation of this information to patients remains a challenge.

Radiologists and dentists often rely on verbal explanations supplemented with printed or digital images to convey diagnostic findings and treatment planning options. These methods, however, are limited in their ability to effectively communicate complex dental conditions to patients who may lack the medical knowledge to fully understand radiographic images. The communication process is frequently hindered by the technical complexity of the images, resulting in potential misunderstandings that may impact patient decision-making and adherence to recommended treatment plans.

Many dental practitioners have integrated digital imaging systems that allow radiologists to view, enhance, and annotate images directly on a computer. This enables more precise identification of dental issues and the ability to highlight specific areas of concern. While these digital tools are helpful for professionals, patients often find it difficult to interpret these annotated images without additional context.

There remains a significant gap in patient comprehension of dental radiological information and treatment planning. Patients often struggle with understanding technical jargon, interpreting radiographic images, and grasping the implications of diagnostic findings for their oral health. Consequently, there is a need for more intuitive, patient-friendly methods of presenting diagnostic information and treatment planning options.

### SUMMARY

The present invention relates to the presentation of diagnostic and treatment planning information to patients. More specifically, the present invention relates to the presentation of radiographic diagnostic information and dental treatment planning options to dental patients.

In one aspect, there is provided a computer system having a display, at least one data storage device configured to access a volumetric dentoalveolar model representing a dentoalveolar state of a patient, at least one non-transitory computer-readable medium having instructions stored thereon which, when executed by at least one computer processor, cause the at least one computer processor to: access the volumetric dentoalveolar model, provide a graphical user interface in data exchange connection with the volumetric dentoalveolar model, the graphical user interface configured to include a plurality of data structures aligned in form and position with corresponding dentoalveolar structures of the volumetric dentoalveolar model, display the volumetric dentoalveolar model and the graphical user interface on the display, in response to user input, select one data structure of the plurality of data structures, and, update the graphical user interface to provide at least one user control configured to adjust at least one of position, orientation, pan and scale of the volumetric dentoalveolar model on the display relative to the one data structure in response to user input to the at least one user control. Any dentoalveolar structure of the corresponding dentoalveolar structures may include a plurality of dentoalveolar structure portions of the volumetric dentoalveolar model, each having a data structure in data exchange connection therewith.

In another aspect, the at least one non-transitory computer-readable medium has further instructions to: update the graphical user interface to include an indication that the one data structure is a selected data structure. The indication may be at least one of a frame surrounding the selected data structure and an outline of contours of the selected data structure.

In another aspect, the at least one non-transitory computer-readable medium has further instructions to: update the graphical user interface to connect in data exchange connection with a reference node positioned relative to the selected data structure, wherein the at least one user control is configured to adjust at least one of position, orientation, pan and scale of the volumetric dentoalveolar model on the display relative to the reference node in response to user input to the at least one user control. The reference node may be positioned one of within the one data structure and externally to the one data structure.

In another aspect, the at least one non-transitory computer-readable medium has further instructions to: update the graphical user interface to provide a second user control to selectively position the reference node relative to the selected data structure.

In another aspect, the at least one non-transitory computer-readable medium has further instructions to at least one of: increase transparency of data structures of the plurality of data structures other than the one data structure relative to the one data structure; and, desaturate color of data structures of the plurality of data structures other than the one data structure relative to the one data structure.

In another aspect, there is provided at least one non-transitory computer-readable medium having instructions stored thereon which, when executed by at least one computer processor, cause the at least one computer processor to: access a volumetric dentoalveolar model representing a dentoalveolar state of a patient, provide a graphical user interface in data exchange connection with the volumetric dentoalveolar model, the graphical user interface configured to include a plurality of data structures aligned in form and position with corresponding dentoalveolar structures of the volumetric dentoalveolar model, display the volumetric dentoalveolar model and the graphical user interface on a display, in response to user input, select one data structure of the plurality of data structures, and, update the graphical user interface to provide at least one user control configured to adjust at least one of position, orientation, pan and scale of the volumetric dentoalveolar model on the display relative to the one data structure in response to user input to the at least one user control. The volumetric dentoalveolar model may be accessed from at least one data storage device having the volumetric dentoalveolar model accessible thereto. Any dentoalveolar structure of the corresponding dentoalveolar structures may include a plurality of dentoalveolar structure portions of the volumetric dentoalveolar model, each having a data structure in data exchange connection therewith.

In one aspect, the non-transitory computer-readable medium further includes instructions to update the graphical user interface to include an indication that the one data structure is a selected data structure. The indication may be at least one of a frame surrounding the selected data structure and an outline of contours of the selected data structure.

In one aspect, the non-transitory computer-readable medium further includes instructions to update the graphical user interface to connect in data exchange connection with a reference node positioned relative to the selected data structure, wherein the at least one user control is configured to adjust at least one of position, orientation, pan and scale of the volumetric dentoalveolar model on the display relative to the reference node in response to user input to the at least one user control. The reference node may be positioned one of within the selected data structure and externally to the selected data structure.

In one aspect, the non-transitory computer-readable medium further includes instructions to update the graphical user interface to provide a second user control to selectively position the reference node relative to the selected data structure.

In one aspect, the non-transitory computer-readable medium further includes instructions to at least one of increase transparency of data structures of the plurality of data structures other than the one data structure relative to the one data structure, and, desaturate color of data structures of the plurality of data structures other than the one data structure relative to the one data structure.

In another aspect, there is provided a method comprising the steps of accessing, via at least one computer processor, a volumetric dentoalveolar model representing a dentoalveolar state of a patient, providing a graphical user interface configured to be in data exchange connection with the volumetric dentoalveolar model, and configured to include a plurality of data structures aligned in form and position with corresponding dentoalveolar structures of the volumetric dentoalveolar model, displaying the volumetric dentoalveolar model and the graphical user interface on a display, in response to user input, selecting a data structure of the plurality of data structures, and, updating the graphical user interface to provide at least one user control configured to adjust at least one of position, orientation, pan and scale of the volumetric dentoalveolar model on the display relative to the one data structure in response to user input to the at least one user control.

In one aspect, the method further includes the step of updating the graphical user interface to include an indication that the one data structure is a selected data structure.

In another aspect, the method further includes the step of updating the graphical user interface to connect in data exchange connection with a reference node positioned relative to the selected data structure, wherein the at least one user control is configured to adjust at least one of position, orientation, pan and scale of the volumetric dentoalveolar model on the display relative to the reference node in response to user input to the at least one user control.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a system, according to one aspect;

FIG. 2 illustrates a graphical user interface (GUI), according to one aspect;

FIG. 3 is a flow chart illustrating a method, according to one aspect;

FIG. 4 illustrates a graphical user interface with a volumetric dentoalveolar model, according to one aspect;

FIG. 5 illustrates a graphical user interface with a volumetric dentoalveolar model, according to one aspect;

FIG. 6 illustrates a graphical user interface with a volumetric dentoalveolar model, according to one aspect;

FIG. 7 illustrates a graphical user interface with a volumetric dentoalveolar model, according to one aspect;

FIG. 8 illustrates a graphical user interface with a volumetric dentoalveolar model, according to one aspect;

FIG. 9 illustrates a graphical user interface with a volumetric dentoalveolar model, according to one aspect; and,

FIG. 10 illustrates a graphical user interface with a volumetric dentoalveolar model, according to one aspect.

### DETAILED DESCRIPTION

The present invention relates to the presentation of diagnostic and treatment planning information to patients. More specifically, the present invention relates to the presentation of radiographic diagnostic information and dental treatment planning options to dental patients.

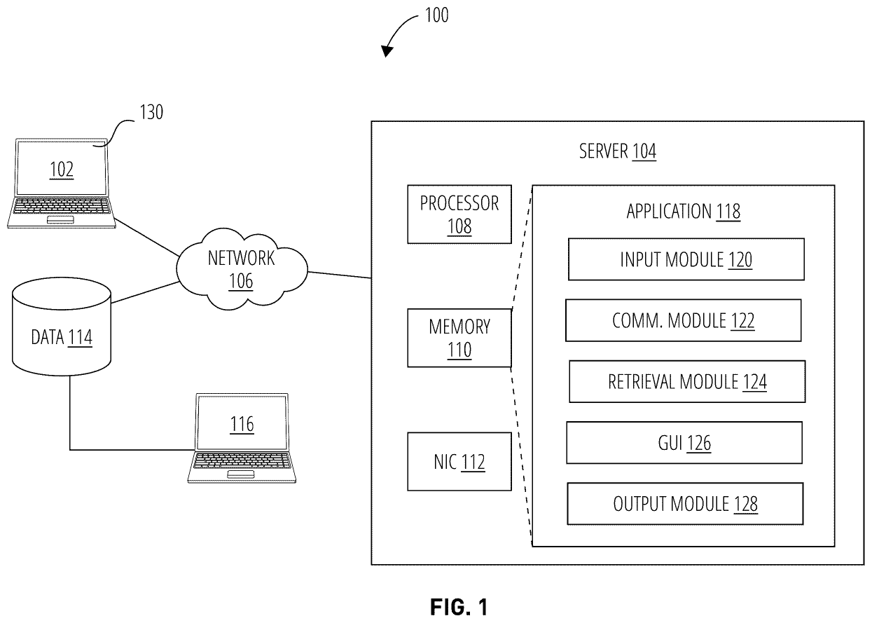

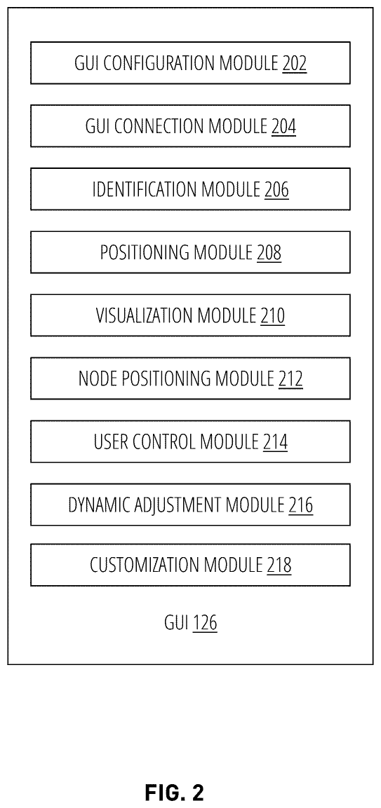

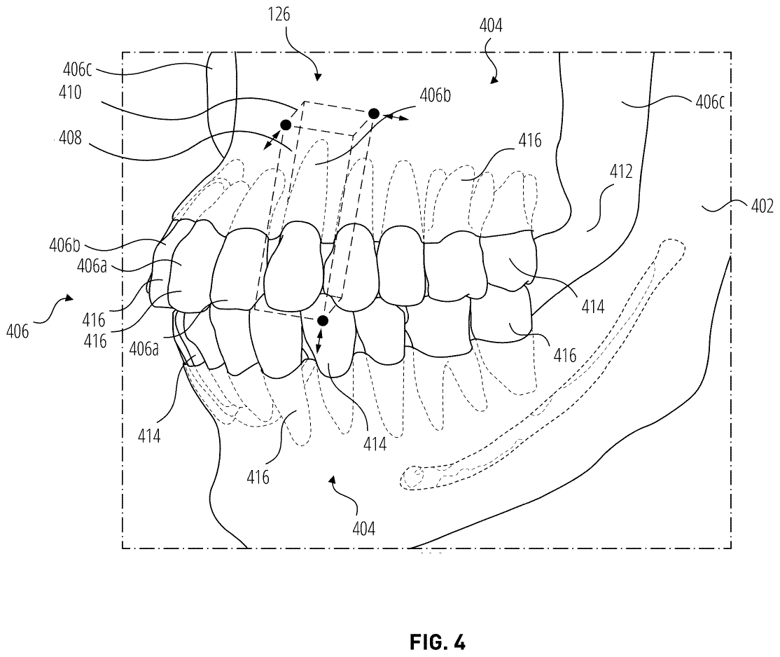

FIG. 1 is a block diagram illustrating an example system 100, according to one aspect. The system 100 includes at least one client device 102 which may comprise one or more computers accessible by one or more users, such as a patient. Client device 102 may be any suitable device (e.g., a laptop, a smart phone, a tablet, a wearable device, a blade server, etc.). Client device 102 may include a memory and a processor for, respectively, storing and executing one or more modules. The memory may include one or more suitable storage media such as a magnetic storage device, a solid-state drive, random access memory (RAM), etc. Aspects of FIG. 1 and FIG. 2 are discussed herein with additional reference to FIG. 4.

The example aspect of FIG. 1 further includes at least one server 104. Server 104 may include a single, standalone server or may include a plurality of servers in data exchange communication with one another, such as in a server system environment. In further aspects, server 104 may be implemented as one or more cloud-based servers, such as a cloud-based computing platform. In some aspects, server 104 may perform the functionalities as discussed herein as part of a “cloud” network or may otherwise communicate with other hardware or software components within one or more cloud computing environments to send, retrieve, or otherwise analyze data or information described herein. For example, in aspects of the present techniques, the cloud computing environment may comprise a customer on-premise computing environment, a multi-cloud computing environment, a public cloud computing environment, a private cloud computing environment, and/or a hybrid cloud computing environment.

Client devices 102 and server 104 are connected by way of network 106. Network 106 may comprise any suitable network. Generally, the network enables bidirectional communication between client device 102 and server 104.

In the aspect of FIG. 1, there is also connected to server 104, via network 106, data storage device 114 upon which may be stored data required for operation of the system 100 as described herein. Data storage device 114 may have stored thereon patient data, such as one or more volumetric dentoalveolar models 402 (FIG. 4) representing a dentoalveolar state of the patient. The patient data may be accessed or retrieved as needed by server 104 and/or applications or modules stored thereon in response to execution of computer-executable instructions as described herein.

In one aspect, the volumetric dentoalveolar model 402 may be a DICOM (Digital Imaging and Communications in Medicine) file, which is a specialized file format widely used in the field of medical imaging. DICOM files typically contain medical images, such as image data from medical or dental scanning operations. DICOM files may be use in advanced visualization techniques, such as 3D reconstruction from scan data. DICOM files may also include metadata. Metadata may contain information about the patient (e.g., name, ID, date of birth), imaging procedure (e.g., type of scan, parameters), and equipment used (e.g., device manufacturer, settings). Metadata makes the DICOM file self-contained and useful for clinical and diagnostic purposes. DICOM also includes a communication protocol that allows devices, such as imaging scanners, workstations, and PACS (Picture Archiving and Communication Systems), to exchange files seamlessly. Physicians and radiologists may use DICOM files to view and analyze diagnostic images for various conditions.

“Dentoalveolar state” refers to the condition of the dental and alveolar structures of a patient, including the teeth and the alveolar bone that supports them. This state is represented through volumetric dentoalveolar models stored in a data storage device, which can be accessed and analyzed by various components of the system, such as the server, diagnostician system, and software modules. The dentoalveolar state provides a detailed and accurate representation of the patient's dental anatomy, facilitating diagnostic and treatment processes.

Diagnostician system 116 may be connected directly with data storage device 114, as shown in the aspect of FIG. 1. In another aspect, diagnostician system 116 may be connected with data storage device 114 via network 106. Diagnostician system 116 is a system of one or more computers, diagnostic equipment and other components used by a diagnostician for the purpose of generating and analyzing patient data to be stored on data storage device 114 and/or accessed by server 104.

Server 104 may include a processor 108, memory 110, a network interface controller (NIC) 112. NIC 112 may include any suitable network interface controller(s), and may communicate over network 106 via any suitable wired and/or wireless connection. Server 104 may include one or more input devices (not depicted) and may include one or more devices for allowing a user to enter inputs (e.g., data) into server 104. For example, the input device may include a keyboard, a mouse, a microphone, a camera, etc. NIC may include one or more transceivers that may be used in receipt and transmission of data via external/network ports connected to network.

Processor 108 may include one or more suitable processors (e.g., central processing units (CPUs) and/or graphics processing units (GPUs)). Processor 108 may be connected to memory 110 via a computer bus (not shown) responsible for transmitting electronic data, data packets, or otherwise electronic signals to and from processor 108 and memory 110 in order to implement or perform the machine readable instructions, methods, processes, or elements, as illustrated, or described herein. Processor 108 may interface with memory 110 via a computer bus to execute an operating system (OS) and/or computing instructions contained therein, and/or to access other services/aspects. For example, processor 108 may interface with memory 110 via the computer bus to create, read, update, delete, or otherwise access or interact with the data stored in memory 110 and/or data storage device 114.

Memory 110 may include one or more forms of volatile, non-volatile, fixed and removable memory. Memory 110 may store an operating system (OS) capable of facilitating the functionalities, applications, methods, computer-executable instructions, or software as discussed herein.

In general, a computer program or computer based product, application, or code may be stored on a computer usable storage medium, or tangible, non-transitory computer-readable medium (e.g., standard random access memory (RAM), an optical disc, a universal serial bus (USB) drive, or the like), such as memory 110, having such computer-readable program code or computer-executable instructions embodied therein, wherein the computer-readable program code or computer-executable instructions may be installed on or otherwise adapted to be executed by a processor, such as processor 108 (e.g., working in connection with the respective operating system in memory), to facilitate, implement, or perform the machine readable instructions, methods, processes, or elements, as illustrated, or described herein. Memory 110 may store a plurality of modules, which provide one or more sets of computer-executable instructions. In the aspect shown in FIG. 1, the plurality of modules is in the form of an application 118. In another aspect, the plurality of modules may be in the form of a plurality of applications or other operations cooperating to provide the one or more sets of computer-executable instructions.

In the aspect shown in FIG. 1, application 118 includes an input module 120, a graphical user interface (GUI) 126 and an output module 128.

Input module 120 includes a set of computer-executable instructions for implementing communication functions. Input module 120 may be in communication with or have embedded therein a communication module 122 configured to communicate (e.g., send and receive) data via one or more external/network port(s) to one or more networks or local terminals, such as network 106 and/or client device 102 (for rendering or visualizing) as described herein. In some aspects, server 104 may include a client-server platform technology responsive for receiving and responding to electronic requests.

Retrieval module 124 retrieves data, via input module 120, from data storage device 114, client device 102 or diagnostician system 116, as required. In one aspect, data which is retrieved by retrieval module 124 includes volumetric dentoalveolar model 402 of a patient, for presentation to a user via client device 102 with GUI 126.

GUI 126 facilitates user interaction and visualization capabilities within the system 100. GUI 126 includes a set of computer-executable instructions, which, when executed by at least one processor, cause the processor to couple or connect GUI 126 in data exchange communication with a volumetric dentoalveolar model 402 and provide one or more controls by which a user, such as a patient, may make user inputs for the purpose of updating visual presentation of volumetric dentoalveolar model 402. GUI 126 is discussed in further detail with respect to FIG. 2. GUI 126 includes a plurality of data structures 406 (FIG. 4) aligned in form and position with corresponding dentoalveolar structures 404 of volumetric dentoalveolar model 402.

Although data structures 406 of GUI 126 are aligned in form and position with the corresponding dentoalveolar structures 404 of volumetric dentoalveolar model 402, there may be some minor differences between the form and position of the data structure 406 of GUI 126 and volumetric dentoalveolar model 402 as they are distinct entities. Therefore, the form and position of the data structure 406 for a corresponding dentoalveolar structure may not necessarily precisely match the form of the corresponding dentoalveolar structure, without departing from the operability and functionality of the invention as described herein.

Output module 128 serves various functions within the system 100. In one aspect, output module 128, upon receiving instructions from GUI 126, causes volumetric dentoalveolar model 402 and GUI 126 to be output to a display 130. The display may be a standalone display or may be a display that is integrated or coupled with client device 102 so that volumetric dentoalveolar model 402 and GUI 126 are displayed to a user. In some aspects, the output module 128 is configured to incorporate GUI 126 wherein the output module 128 and GUI 126 may operate collectively.

As shown in FIG. 2, GUI 126 includes a plurality of modules which provide a set of computer-executable instructions for execution by at least one processor, such as processor 108. Although the plurality of modules of FIG. 2 are shown as being separate and distinct from one another, separate modules may be combined into singular modules with divided functions or may be embedded within one another to provide the functionality described herein to GUI 126.

In general, the word “module,” as used herein, refers to a collection of software instructions, possibly having entry and exit points, written in a programming language, such as, for example, Java, Lua, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware devices (such as processors and CPUs) may be comprised of connected logic units, such as gates and flip-flops, and/or may be comprised of programmable units, such as programmable gate arrays or processors. The modules or computing device functionality described herein are preferably implemented as software modules but may be represented in hardware devices. Generally, the modules described herein refer to software modules that may be combined with other modules or divided into sub-modules despite their physical organization or storage.

GUI configuration module 202 is responsible for displaying the graphical elements of GUI 126 to client device 102 and rendering visual components of GUI 126. Configuration module 202 draws or renders controls such as buttons, text fields, menus, sliders, checkboxes, icons and other graphical elements and provides them to a display for a user, by way of client device 102, for example. Configuration module 202 detects inputs from user peripherals, such as a mouse, keyboard or touch screen, or other interactions made by the user via client device 102. Configuration module 202 also updates GUI 126 when the underlying data changes, such as when a new control or notification appears. Preferably, such updating is automatic and in real time. Updates to GUI 126 are preferably initiated by configuration module 202 and performed by visualization module 210. In another aspect, visualization module 210 is an embedded component of configuration module 202 and not a distinct module, as shown in the aspect of FIG. 2.

GUI connection module 204 is responsible for integrating GUI 126 with data retrieved from data storage device 114, such as volumetric dentoalveolar model 402. That is, connection module 204 couples GUI 126 in data exchange communication with volumetric dentoalveolar model 402. GUI connection module 204 is also responsible for integrating volumetric dentoalveolar model 402 with controls and other interactive elements of GUI 126 based on conditions or user inputs. Connection module 204 acts as a mediator between the data and GUI 126, which updates or tailors displayed content by dynamically adding or adjusting control elements in GUI 126 based on the current context or user input received at GUI 126 from client device 102 via input module 120. Connection module 204 adds, removes, customizes or updates control elements, such as buttons, text fields, menus, sliders, checkboxes, icons and other graphical elements dynamically based on changes in the data implemented by way of GUI 126 or based on preferences of the user. In another aspect, connection module 204 may modify GUI 126 based on the state of the application 118, user role, or permissions. In another aspect, connection module 204, may integrate data elements with control features, such as populating drop-down lists with data retrieved from data storage device 114 or may dynamically adjust layout of GUI 126, data such as volumetric dentoalveolar model 402 or controls related thereto based on changes in volumetric dentoalveolar model 402 or in response to external inputs.

GUI 126 is configured to include a plurality of data structures 406 which are aligned in form and position with corresponding dentoalveolar structures 404 (FIG. 4) of volumetric dentoalveolar model 402. Data structure identification module 206 cooperates with configuration module 202 and positioning module 208 to provide a plurality of data structures 406 and to position each data structure 406 relative to at least one dentoalveolar structure of volumetric dentoalveolar model 402, and to select one data structure 406 of the plurality of data structures 406 in response to user input. Identification module 206 may further cooperate with connection module 204 for positioning of user controls and other GUI 126 elements relative to the volumetric dentoalveolar model.

Positioning of data structures 406 within GUI 126 may be determined based on any suitable criteria. For example, data structure positioning may be determined based on the anatomical structure of volumetric dentoalveolar model 402. These criteria may include specific landmarks or reference points within volumetric dentoalveolar model 402 that are used to define the boundaries and positions of data structures 406. Suitable algorithms or user-defined settings may be employed to ensure that data structures 406 are accurately positioned relative to the corresponding modeled dentoalveolar structures of volumetric dentoalveolar model 402. For example, data structures 406 may be positioned based on the alignment of teeth, the curvature of the jaw, or other anatomical features represented within volumetric dentoalveolar model 402.

On selection of the one data structure 406 of the plurality of data structures 406, identification module 206 may communicate with configuration module 202 and/or visualization module 210 to update GUI 126 to reflect the selection of the one data structure 406. In another aspect configuration module 202 communicates with visualization module 210 to provide an indication that the one data structure 406 is a selected data structure 408 (FIG. 4).

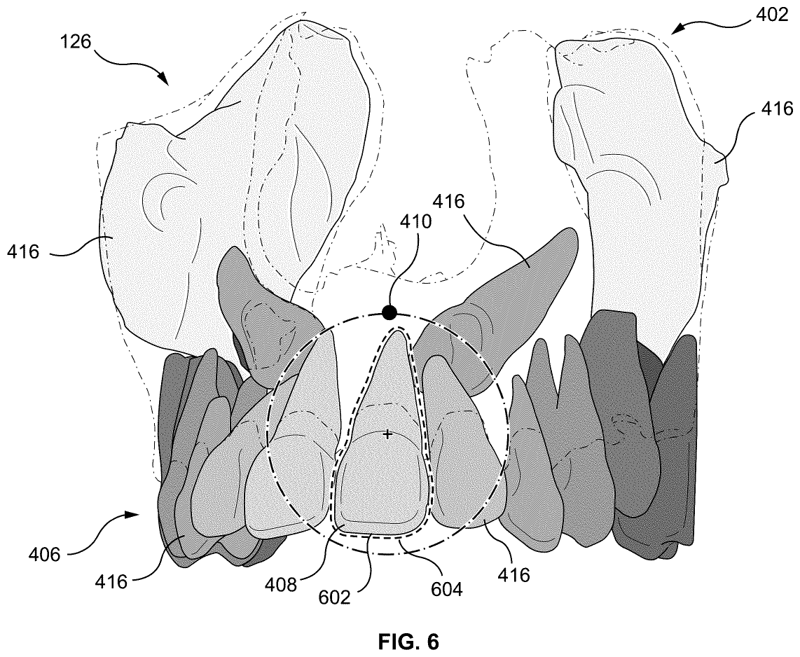

Preferably, the indication is an outline 604 of the contours 602 (FIG. 6) of selected data structure 408 in a highlighted color that makes selected data structure 408 more visually apparent to the user of client device 102. Preferably, the outline is updated to reflect the visible contour of selected data structure 408 as volumetric dentoalveolar model 402 is repositioned via GUI 126, as further described hereinafter. In another aspect, the indication may be a frame 410 (FIG. 4) or three-dimensional box surrounding selected data structure 408. While frame 410 may be less precise than the outline of the contour of selected data structure 408, it is a less computationally intensive option and is less demanding to render than the outline of the contour of selected data structure 408. The use of such visual indications provides further clarity to the user viewing the information presented by system 100.

While identification module 206 is shown as a single module in FIG. 2, in another aspect, the identification module 206 may include a set of separate modules for providing the plurality of data structures 406, positioning each data structure 406 relative to at least one structure of volumetric dentoalveolar model 402 and selecting one data structure of the plurality of data structures 406. Moreover, while the selection is described herein as being made by a user, the user need not be human in all cases. Rather, the selection may be made by a machine learning or artificial intelligence agent acting on instructions from a human user or on behalf of a human user.

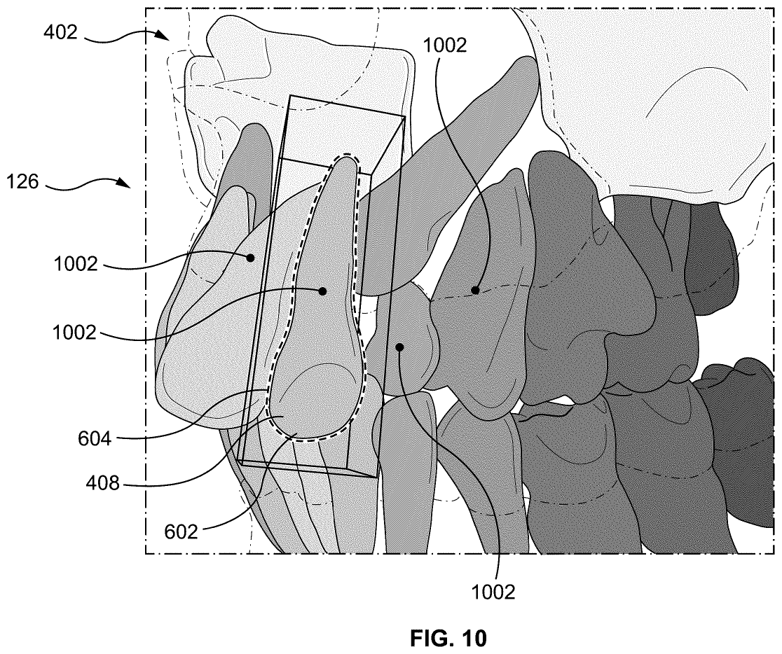

GUI 126 further includes node positioning module 212. Node positioning module 212 positions a reference node 1002 (FIG. 10) relative to each data structure 406 and connects reference node 1002 in data connection to the corresponding data structure 406. Node positioning module 212 communicates with configuration module 202 and a user control module 214 to update GUI 126 to provide at least one user control. The at least one user control is configured to adjust at least one of position, orientation, pan and scale of volumetric dentoalveolar model 402 on the display relative to reference node 1002 in response to user input to the at least one user control. Preferably, GUI 126 is updated to include individual controls for each of position, orientation, pan and scale of volumetric dentoalveolar model 402. Controls for interacting with or changing other aspects related to the position and orientation or visualization of volumetric dentoalveolar model 402 may included into GUI 126 via user control module 214 and configuration module 202.

In one preferred aspect, the system is configured to enable a user to interact with a volumetric image using an input device such as a mouse, where different manipulations, such as rotation, panning, and zooming, may be performed based on specific input actions. These controls provide an invisible and unobtrusive interface configured to allow a user, such as a patient, to focus entirely on the visualization of the volumetric dentoalveolar model 402.

In one preferred aspect, to rotate the volumetric image, the user may hold the left mouse button while moving the mouse in the desired direction of rotation. This input is translated into corresponding rotational adjustments of the volumetric dentoalveolar model 402 and GUI 126 around its axes. Panning the volumetric dentoalveolar model 402 and GUI 126 may be accomplished by holding the right mouse button and moving the mouse, enabling translation of the volumetric dentoalveolar model 402 and GUI 126 along the x- and y-axes. Zooming in and out may be achieved by rotating the mouse wheel, which adjusts the scale of the volumetric dentoalveolar model 402 and GUI 126 incrementally or decrementally to bring areas of interest into or out of focus or to increase or decrease their relative scale. By combining these actions, system 100 offers a fluid and natural interaction model for exploring complex volumetric datasets without the need for obtrusive on-screen graphical controls.

In another aspect, the at least one user control may include on-screen graphical controls such as buttons, sliders, and other interactive elements for controlling display of the volumetric dentoalveolar model 402 and GUI 126.

In one preferred aspect, GUI 126 includes a dynamic adjustment module 216 which is configured to adjust, preferably in real time, visual elements such as brightness, contrast, color balance, sharpness, and scaling to enhance or optimize the visual presentation or appearance of volumetric dentoalveolar model 402 in response to user inputs or contexts arising from user interaction with GUI 126. In another aspect, this process can be automated using algorithms that analyze volumetric dentoalveolar model 402 and GUI 126 to adjust settings for better visibility, clarity, or aesthetic appeal. Dynamic adjustment module 216 may be configured to allow users to adjust visual settings, such as opacity or focus, of specific dentoalveolar structure 404 and/or dentoalveolar structure portion 902 or of the entire volumetric dentoalveolar model 402. Adapting image attributes dynamically improves the experience of the patient when using GUI 126 in association with volumetric dentoalveolar model 402 by optimizing for varying situations or circumstances.

Dynamic adjustment module 216 may also be responsive to the user control module 214 to provide the user with controls which may be used for user input to adjust visual settings of volumetric dentoalveolar model 402.

Customization module 218 provides at least one customization control, responsive to user input, which allows a user to customize visual components of GUI 126. For example, a user may input commands to customization module 218 to increase line thickness of the indication around selected data structures 408 or may change the color thereof to increase contrast or highlighting relative to surrounding non-selected data structures 416 and to make selected data structure 408 more apparent.

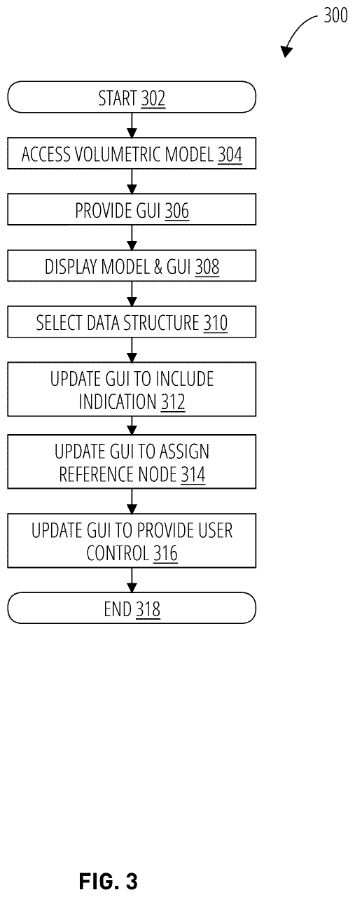

FIG. 3 is a flow chart for an example method 300 according to one aspect. Preferably, at least one non-transitory computer-readable medium has stored thereon a set of instructions which, when executed by at least one computer processor, cause the at least one computer processor to execute the steps of method 300. The at least one processor may be one or more of the processors of system 100.

The method begins at block 302. At block 304, a volumetric dentoalveolar model representing a dentoalveolar state of a patient is accessed. Preferably, volumetric dentoalveolar model is accessed from data storage device 114 having volumetric dentoalveolar model 402 accessible thereto. The retrieval module 124 retrieves this data from a storage device such as data storage device 114 via input module 120, shown in FIG. 1. Specific protocols, such as secure data transmission and validation checks, may be used to ensure the accuracy and integrity of the retrieved volumetric dentoalveolar model.

At block 306, GUI 126 is provided wherein GUI 126 is configured to be in data exchange connection with volumetric dentoalveolar model 402. GUI 126 is further configured to include a plurality of data structures 406 aligned in form and position with corresponding dentoalveolar structures 404 of volumetric dentoalveolar model 402. This configuration is managed by GUI configuration module 202, which displays graphical elements and manages visual components. In one aspect, the data structures are positioned based on predefined criteria or user inputs and ensure accurate representation of the dentoalveolar structures in volumetric dentoalveolar model 402.

At block 308, volumetric dentoalveolar model 402 and GUI 126 are displayed on a display 130. Displaying GUI 126 on a display 130 may further include the steps of rendering the graphical elements and controls of GUI 126, updating GUI 126 based on user interactions or data changes, and outputting GUI 126 to a display device. As previously described, display 130 can be a standalone display or integrated with client device 102. Technologies such as high-resolution screens and touch interfaces may be used for better visualization and interaction.

At block 310, in response to user input, one data structure 406 of the plurality of data structures 406 is selected. The system 100 detects and processes user input through input devices such as a mouse, keyboard, or touch screen. The user selects one data structure 406 from the plurality of data structures 406 using these input devices. The identification module 206 cooperates with configuration module 202 and connection module 204 to manage this selection process.

At block 316, GUI 126 is updated to provide at least one user control configured to adjust at least one of position, orientation, pan and scale of volumetric dentoalveolar model 402 on the display 130 relative to reference node 1002 in response to user input to the at least one user control. The user control module 214 manages these controls and ensures that the system 100 responds accurately to user input. Dynamic adjustment module 216 may also adjust visual elements in real-time based on user interactions.

Method 300 may include a further step shown at block 312 wherein, prior to the step described at block 316, GUI 126 is updated to include an indication that the one data structure 406 is a selected data structure 408. In one aspect, indication that the one data structure 406 is selected is by highlighting the one data structure 406 with an outline or frame. The identification module 206 communicates with configuration module 202 and visualization module 210 to update GUI 126.

Method 300 may include a further step shown at block 314, wherein, prior to the step described at block 316, GUI 126 is updated to connect in data exchange connection with a reference node 1002 (FIG. 10) positioned relative to selected data structure 408, wherein the at least one user control is configured to adjust at least one of position, orientation, pan and scale of the volumetric dentoalveolar model on the display relative to reference node 1002 in response to user input to the at least one user control. Reference node 1002 is positioned relative to selected data structure 408 by node positioning module 212. In some aspects, reference node 1002 may be positioned within selected data structure 408 and in other aspects, reference node 1002 may be positioned externally to selected data structure 408.

At block 318, method 300 ends. System 100 ensures that all user inputs have been processed, GUI 126 is updated to reflect the final state, and any necessary data is stored or transmitted.

In FIG. 4, there is shown an exemplary volumetric dentoalveolar model 402 with GUI 126. Volumetric dentoalveolar model 402 includes a plurality of dentoalveolar structures 404, which are modeled components of the greater volumetric dentoalveolar model 402. The plurality of dentoalveolar structures 404 may be identified by any suitable manner, such as by using segmentation algorithms that detect the boundaries of the dentoalveolar structures 404 making up volumetric dentoalveolar model 402.

GUI 126 is connected in data exchange connection with volumetric dentoalveolar model 402, GUI 126 being configured to include a plurality of data structures 406 aligned in form and position with corresponding dentoalveolar structures 404 of volumetric dentoalveolar model 402. Data structures 406 may represent groups of modeled dentoalveolar structures 404, as shown with respect to data structures 406a, 406b and 406c, among others. Data structures 406 may also represent single modeled dentoalveolar structures 404, as shown with respect to data structure 406d. Data structures 406 may be grouped according to any predetermined criteria or may be grouped in a manner determined by a radiologist or dentist making use of system 100. Accordingly, it is not required that data structures 406 be grouped according to any particular anatomical criteria, though this may be desired depending on the needs of the radiologist, dentist or patient.

GUI 126 identifies a selected data structure 408 by way of an identification in the form of frame 410 surrounding a single modeled tooth. Frame 410 may be a bounding box, for example. In one aspect, frame 410 is generated around selected data structure 408 using algorithms that detect the boundaries of selected data structure 408.

GUI 126 also identifies a plurality of non-selected data structures 416, each corresponding to one or more modeled structures of the volumetric dentoalveolar model 402 of a patient other than selected data structure 408. Each of the non-selected data structures 416 is identified using algorithmic or manual methods similar to those for identifying selected data structure 408. Non-selected data structure 416 may have coloration which is distinct from that of selected data structure 408 and from one another. In the exemplary aspect of FIG. 4, non-selected data structures 416 include the modeled maxilla 412 or other modeled teeth 414, among others.

In one aspect, frame 410 is customizable in terms of color, thickness, and style. Users can adjust these settings through GUI 126, which provides options for customizing the appearance of frame 410. This allows users to choose a color that contrasts with the surrounding regions or data structures 406, adjust the thickness for better visibility, and select a style that suits their preferences.

In addition to distinct coloration, selected data structure 408 is also more opaque than the non-selected data structures 416. The non-selected data structures 416 are more transparent than selected data structure 408. In another aspect, color in the non-selected data structures 416 may be desaturated relative to color of the selected data structures 408. Desaturation of the non-selected data structures 416 visually de-emphasizes the non-selected data structures 416, allowing for easier viewing and inspection of selected data structure 408.

Thereby, non-selected data structures 416 are identified and distinguished from selected data structure 408 using visual indicators such as distinct coloration and transparency. In one aspect, each non-selected data structure 416 is assigned a unique color that is different from selected data structure 408 and other non-selected data structures 416. Additionally, the non-selected data structures 416 may be made more transparent to visually de-emphasize them.

The opacity of selected data structure 408 and non-selected data structures 416 is controlled through dynamic adjustment module 216. Dynamic adjustment module 216 provides settings and controls that allow users to adjust the opacity levels of different data structures 406. Users can increase the opacity of selected data structure 408 to make it more prominent and decrease the opacity of non-selected data structures 416 to de-emphasize them. In another aspect, opacity of selected data structures 408 and non-selected data structures 416 may be controlled by a suitable algorithm.

Desaturation of the non-selected data structures 416 may be implemented manually or by using algorithms that reduce the color intensity of the non-selected data structures 416. The algorithms adjust the color balance and saturation levels to make the non-selected data structures 416 appear more muted. This visual effect helps to emphasize selected data structure 408 by making the non-selected data structures 416 less visually prominent than selected data structure 408.

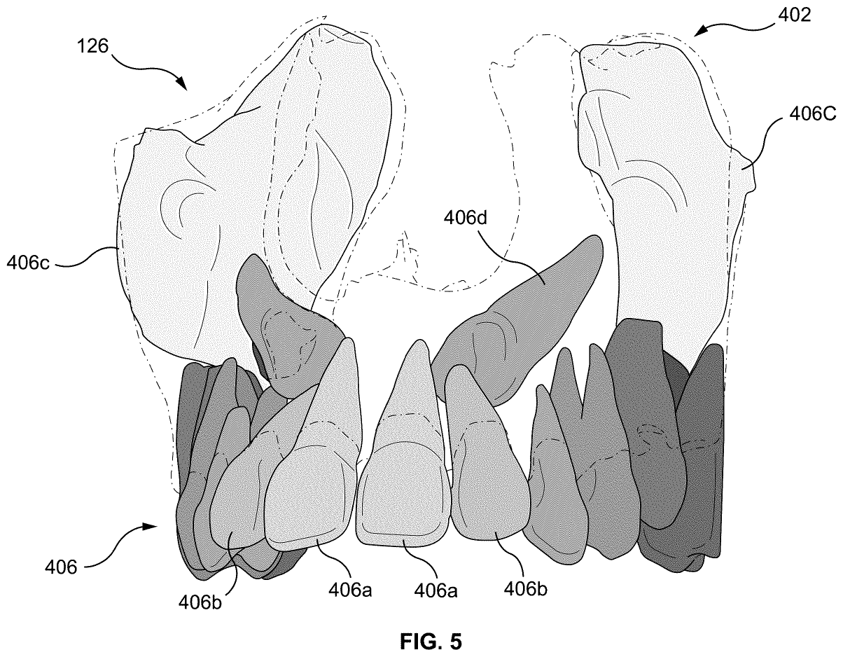

In FIG. 5, there is shown an exemplary volumetric dentoalveolar model 402 which includes a plurality of data structures 406 including data structures 406a, 406b, 406c and data structure 406d which is coupled with a modeled tooth that has grown in an atypical direction. Accompanying volumetric dentoalveolar model 402 is GUI 126. In the aspect of FIG. 5, none of data structures 406 are selected data structures 408. Accordingly, no indication is presented by GUI 126.

In one preferred aspect, algorithms analyze volumetric dentoalveolar model 402 to identify contours 602 of each data structure 406. Once a data structure 406 is identified by GUI 126 as selected data structure 408, outline 604 is applied to the contour 602 of selected data structure 408. Outline 604 can be in any suitable color or thickness. Preferably, outline 604 is a thick line, which may be black, grayscale or colored, which contrasts with the colors of nearby or surrounding non-selected data structures 416. Thereby, selected data structure 408 is made more visually apparent relative to other areas of volumetric dentoalveolar model 402.

A frame 410 may also be drawn around the selected data structures 408 with contours 602 to visually highlight the selected data structures 408 further. Frame 410 may be drawn by input from a user, such as a diagnostician or dentist or may be drawn automatically by a suitable algorithm or software module. In the aspect shown in FIG. 6, frame 410 is a circle rather than a rectangular box, as shown in FIG. 4.

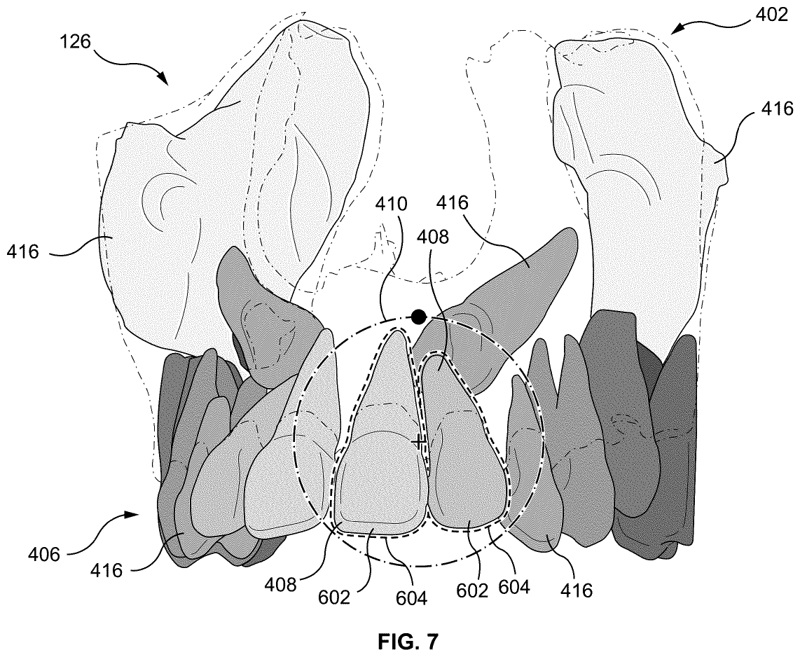

In FIG. 7, it is shown that a second data structure 406 was selected. The second selected data structure 408 is provided with an indication in the form of an outline 604 of its contour 602. Frame 410 encircles both of the selected data structures 408 and is enlarged as compared to its representation in FIG. 6.

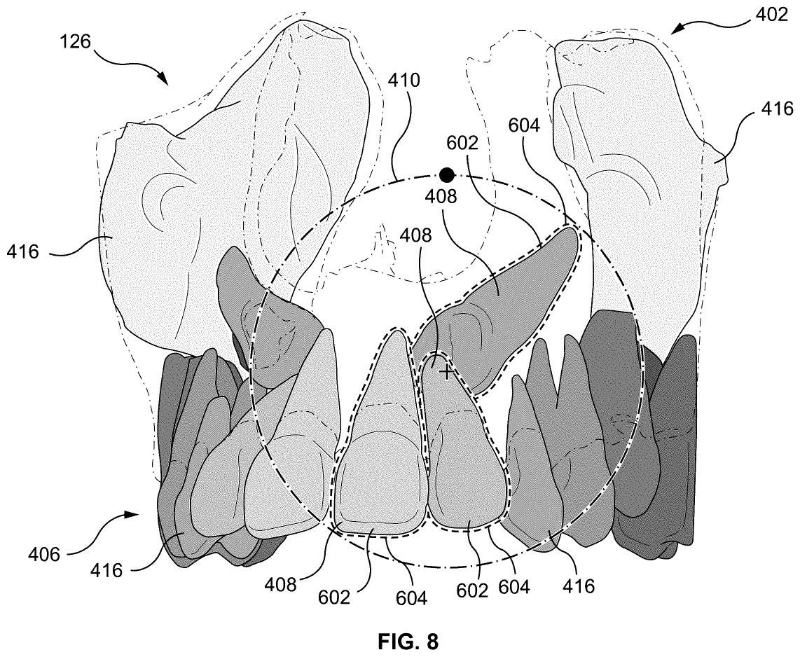

In FIG. 8, it is shown that a third data structure 406 was selected. The third selected data structure 408 is provided with an indication in the form of an outline 604 of its contour 602. Frame 410 encircles all three of the selected data structures 408 and is enlarged as compared to its representation in FIG. 7.

Accordingly, GUI 126 may be updated in response to user inputs to indicate user selection of any number of data structures 406 with related indications being updated with changes to user selections. The indications may include frame 410 and/or outline 604 with frame 410 and outline 604 also updating in response to changes in user selection of data structures 406. Thereby, volumetric dentoalveolar model 402 and GUI 126 provide an adaptive and up to date representation which facilitates patient comprehension of dental radiological information and treatment planning.

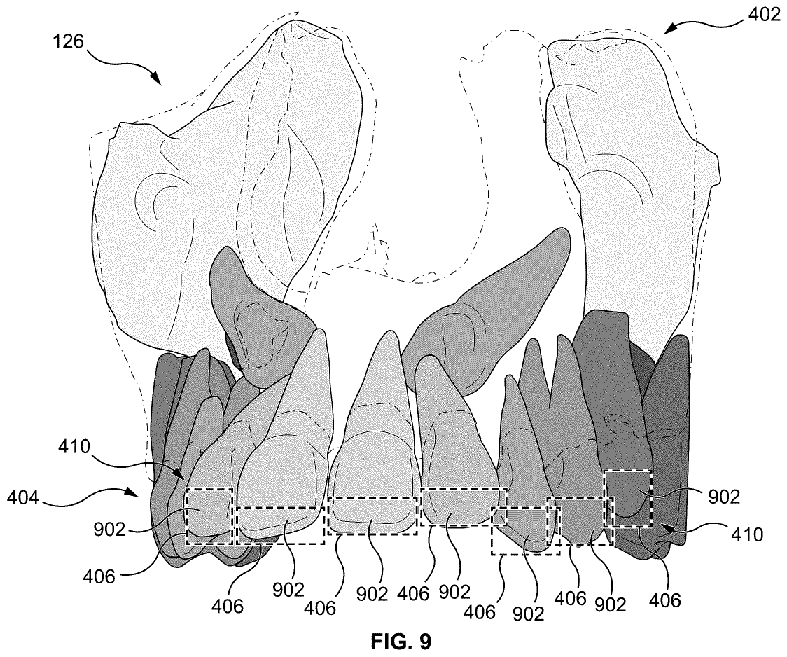

In the aspect shown in FIG. 9, GUI 126 is configured to include a data structure 406 aligned in form and position with a corresponding dentoalveolar structure 404. Any dentoalveolar structure 404 of the corresponding dentoalveolar structures 404 may include a plurality of dentoalveolar structure portions 902 of volumetric dentoalveolar model 402, each having a data structure 406 in data exchange connection therewith. In some instances, a radiologist or dentist may wish to indicate a condition or treatment that applies to parts of a plurality of teeth, such as only the crowns or roots or to areas of multiple teeth affected by decay.

Each dentoalveolar structure portion 902 may be identified in the same manner as previously described for identification of data structures 406. That is, identification of dentoalveolar structure portions 902 may be made using suitable algorithms or software that analyze volumetric dentoalveolar model 402 to detect distinct anatomical features. These algorithms may use criteria such as the shape, size, and position of the modeled teeth or other dental structures to determine the boundaries of each dentoalveolar structure portion 902. A user may also manually input criteria for identifying dentoalveolar structure portions 902. For example, software may be used to identify dentoalveolar structure portions 902 generally and a user may make adjustments to dentoalveolar structure portions 902 for accuracy or specificity.

In the aspect of FIG. 9, there is shown a data structure 406, which may be a selected data structure 408, which includes a plurality of dentoalveolar structure portions 902 representing the crowns of a plurality of modeled teeth. As with previously described aspects, the dentoalveolar structure portions 902 are provided with an indication, such as frame 410. In other aspects, the indication may be an outline 604 aligned with contours 602 of the dentoalveolar structure portion 902. The non-selected data structures 416 of each of the modeled teeth may be de-emphasized in the manner previously described herein.

In another aspect, selected data structure 408 may include at least one whole dentoalveolar structure 404 and at least one dentoalveolar structure portion 902. Accordingly, GUI 126 may associate data structures 406 with both whole and partial objects identified in volumetric dentoalveolar model 402. GUI 126 is highly adaptable for visualization of specific features of volumetric dentoalveolar model 402. For example, a single tooth and a segment of the modeled maxilla 412 out of which the tooth projects may be identified as selected data structure 408.

The arrangement of data structures 406 and dentoalveolar structure portions 902 within GUI 126 is managed by the identification module 206 in cooperation with configuration module 202 and positioning module 208. These modules work together to position each data structure 406 and dentoalveolar structure portion 902 relative to the modeled dentoalveolar structures 404 and update GUI 126 based on user inputs and data changes.

As with the aspects previously described, the dentoalveolar structure portions 902 may have visual indicators that are used to distinguish dentoalveolar structure portions 902 from one another and from dentoalveolar structure portions 902 defining selected data structure 408. Visual indicators such as distinct coloration, opacity levels, and outlines may be used to distinguish the dentoalveolar structure portions 902 from one another and from selected data structure 408. In some aspects, each dentoalveolar structure portion 902 may be assigned a unique color or pattern to make it visually distinct. Additionally, selected data structure 408 and any dentoalveolar structure portions 902 may be highlighted with a frame 410 or outline 604 to emphasize those components.

In another aspect, dynamic adjustment module 216 allows users to make real-time adjustments to enhance the visibility and clarity of the dentoalveolar structure portions 902 based on user input. For example, dynamic adjustment module 216 may provide controls within GUI 126 to customize the visualization of specific dentoalveolar structure portions 902. These controls may include sliders, buttons, and checkboxes that allow users to adjust visual settings such as brightness, contrast, and color balance. Users can also use these controls to select and highlight specific regions or dentoalveolar structure portions 902 within volumetric dentoalveolar model 402.

As shown in FIG. 10, dentoalveolar structure 404 and/or dentoalveolar structure portions 902 may have a reference node 1002 positioned relative thereto. Reference nodes 1002 provide a point of reference about which volumetric dentoalveolar model 402 and GUI 126 together may be magnified, demagnified, panned or otherwise repositioned or reoriented by a user of GUI 126. Once a user selects a data structure 406 via GUI 126, reference node 1002 positioned relative to the dentoalveolar structure 404 and/or dentoalveolar structure portions 902 in data exchange connection with selected data structure 408 becomes the active reference node 1002 about which GUI 126 and volumetric dentoalveolar model 402 may be repositioned and/or reoriented.

Preferably, reference node 1002 is positioned within the corresponding dentoalveolar structure 404, such as where selected data structure 408 is a single tooth or group of teeth or other structures in relatively close proximity to one another. In other aspects, however, reference node 1002 may be positioned externally to the corresponding dentoalveolar structure 404 and/or dentoalveolar structure portions 902. For example, reference node 1002 may be positioned externally to the corresponding dentoalveolar structure 404 and/or dentoalveolar structure portion 902 when the dentoalveolar structure 404 and/or dentoalveolar structure portion 902 includes component parts that are spaced apart, such as the roots or crowns of multiple modeled teeth or spaced apart portions of the alveolar bone.

Reference nodes 1002 may be positioned manually or automatically by way of suitable algorithms capable of locating the reference nodes 1002 relative to the corresponding dentoalveolar structure portions 902. Moreover, system 100 may provide user controls responsive to user input for enabling a user to reposition one or more of the reference nodes 1002 as desired in order to enable new perspective views or repositioning options.

While the invention has been described in terms of specific aspects, it is apparent that other forms could be adopted by one skilled in the art. For example, the methods described herein could be performed in a manner which differs from the aspects described herein. The steps of each method could be performed using similar steps or steps producing the same result, but which are not necessarily equivalent to the steps described herein. Some steps may also be performed in different orders to obtain the same result. Similarly, the apparatuses and systems described herein could differ in appearance and construction from the aspects described herein, the functions of each component of the apparatus could be performed by components of different construction but capable of a similar though not necessarily equivalent function, and appropriate materials could be substituted for those noted. Accordingly, it should be understood that the invention is not limited to the specific aspects described herein. It should also be understood that the phraseology and terminology employed above are for the purpose of disclosing the illustrated aspects, and do not necessarily serve as limitations to the scope of the invention.

What is claimed is:

1. A computer system comprising:
a display;
at least one data storage device having configured to access a volumetric dentoalveolar model representing a dentoalveolar state of a patient;
at least one non-transitory computer-readable medium having instructions stored thereon which, when executed by at least one computer processor, cause the at least one computer processor to:
access the volumetric dentoalveolar model;
provide a graphical user interface in data exchange connection with the volumetric dentoalveolar model, the graphical user interface configured to include a plurality of data structures aligned in form and position with corresponding dentoalveolar structures of the volumetric dentoalveolar model;
display the volumetric dentoalveolar model and the graphical user interface on the display;
in response to user input, select one data structure of the plurality of data structures; and,
update the graphical user interface to provide at least one user control configured to adjust at least one of position, orientation, pan and scale of the volumetric dentoalveolar model on the display relative to the one data structure in response to user input to the at least one user control.

2. The computer system of claim 1, wherein the at least one non-transitory computer-readable medium has further instructions to:
update the graphical user interface to include an indication that the one data structure is a selected data structure.

3. The computer system of claim 2, wherein the indication is at least one of a frame surrounding the selected data structure and an outline of contours of the selected data structure.

4. The computer system of claim 1, wherein the at least one non-transitory computer-readable medium has further instructions to:
update the graphical user interface to connect in data exchange connection with a reference node positioned relative to the selected data structure, wherein the at least one user control is configured to adjust at least one of position, orientation, pan and scale of the volumetric dentoalveolar model on the display relative to the reference node in response to user input to the at least one user control.

5. The computer system of claim 4, wherein the reference node is positioned one of within the selected data structure and externally to the selected data structure.

6. The computer system of claim 4, wherein the at least one non-transitory computer-readable medium has further instructions to:
update the graphical user interface to provide a second user control to selectively position the reference node relative to the selected data structure.

7. The computer system of claim 1, wherein the at least one non-transitory computer-readable medium has further instructions to at least one of:
increase transparency of data structures of the plurality of data structures other than the one data structure relative to the one data structure; and,
desaturate color of data structures of the plurality of data structures other than the one data structure relative to the one data structure.

8. The computer system of claim 1, wherein any dentoalveolar structure of the corresponding dentoalveolar structures may include a plurality of dentoalveolar structure portions of the volumetric dentoalveolar model, each having a data structure in data exchange connection therewith.

9. At least one non-transitory computer-readable medium having instructions stored thereon which, when executed by at least one computer processor, cause the at least one computer processor to:
access a volumetric dentoalveolar model representing a dentoalveolar state of a patient;
provide a graphical user interface in data exchange connection with the volumetric dentoalveolar model, the graphical user interface configured to include a plurality of data structures aligned in form and position with corresponding dentoalveolar structures of the volumetric dentoalveolar model;
display the volumetric dentoalveolar model and the graphical user interface on a display;
in response to user input, select one data structure of the plurality of data structures; and,
update the graphical user interface to provide at least one user control configured to adjust at least one of position, orientation, pan and scale of the volumetric dentoalveolar model on the display relative to the one data structure in response to user input to the at least one user control.

10. The non-transitory computer-readable medium of claim 9, wherein the volumetric dentoalveolar model is accessed from at least one data storage device having the volumetric dentoalveolar model accessible thereto.

11. The non-transitory computer-readable medium of claim 9, further comprising instructions to:
update the graphical user interface to include an indication that the one data structure is a selected data structure.

12. The non-transitory computer-readable medium of claim 11, wherein the indication is at least one of a frame surrounding the selected data structure and an outline of contours of the selected data structure.

13. The non-transitory computer-readable medium of claim 9 further comprising instructions to:
update the graphical user interface to connect in data exchange connection with a reference node positioned relative to the selected data structure, wherein the at least one user control is configured to adjust at least one of position, orientation, pan and scale of the volumetric dentoalveolar model on the display relative to the reference node in response to user input to the at least one user control.

14. The non-transitory computer-readable medium of claim 13, wherein the reference node is positioned one of within the selected data structure and externally to the selected data structure.

15. The non-transitory computer-readable medium of claim 13, further comprising instructions to:
update the graphical user interface to provide a second user control to selectively position the reference node relative to the selected data structure.

16. The non-transitory computer-readable medium of claim 9, further comprising instructions to at least one of:
increase transparency of data structures of the plurality of data structures other than the one data structure relative to the one data structure; and,
desaturate color of data structures of the plurality of data structures other than the one data structure relative to the one data structure.

17. The non-transitory computer-readable medium of claim 9, wherein any dentoalveolar structure of the corresponding dentoalveolar structures may include a plurality of dentoalveolar structure portions of the volumetric dentoalveolar model, each having a data structure in data exchange connection therewith.

18. A method comprising the steps of:
accessing, via at least one computer processor, a volumetric dentoalveolar model representing a dentoalveolar state of a patient;
providing a graphical user interface configured to be in data exchange connection with the volumetric dentoalveolar model, and configured to include a plurality of data structures aligned in form and position with corresponding dentoalveolar structures of the volumetric dentoalveolar model;
displaying the volumetric dentoalveolar model and the graphical user interface on a display;
in response to user input, selecting a data structure of the plurality of data structures; and,
updating the graphical user interface to provide at least one user control configured to adjust at least one of position, orientation, pan and scale of the volumetric dentoalveolar model on the display relative to the one data structure in response to user input to the at least one user control.

19. The method of claim 18, further comprising the step of:
updating the graphical user interface to include an indication that the one data structure is a selected data structure.

20. The method of claim 18, further comprising the step of:
updating the graphical user interface to connect in data exchange connection with a reference node positioned relative to the selected data structure, wherein the at least one user control is configured to adjust at least one of position, orientation, pan and scale of the volumetric dentoalveolar model on the display relative to the reference node in response to user input to the at least one user control.
